(19) United States
(12) Patent Application Publication
(10) Pub. No.: US 2025/0255763 A1
(43) Pub. Date: Aug. 14, 2025

(54) METHOD FOR CONTROLLING AN ACTUATOR SYSTEM OF A MEDICAL DEVICE

(71) Applicant: Loewenstein Medical Technology S.A., Luxembourg (LU)

(72) Inventors: Martin STAHL, Damscheid (DE); Johannes MARTIN, Sonthofen (DE); Carsten RUECH, Brey (DE)

(21) Appl. No.: 19/048,242

(22) Filed: Feb. 7, 2025

(30) Foreign Application Priority Data
Feb. 13, 2024 (DE) 102024103998.1

(51) Int. Cl. A61G 11/00

(52) U.S. Cl. CPC A61G 11/005; A61G 2203/10

(57) ABSTRACT

A method for controlling an actuator system of a medical device comprises receiving a sensor signal, which indicates a deformation, detected by means of a sensor, of an actuating element on actuation thereof by a foot and/or a hand; and generating a control signal for controlling the actuator system using the sensor signal.

### CROSS-REFERENCE TO RELATED APPLICATIONS

The present application claims priority under 35 U.S.C. § 119 of German Patent Application No. 102024103998.1, filed Feb. 13, 2024, the entire disclosure of which is expressly incorporated by reference herein.

### BACKGROUND OF THE INVENTION

### 1. Field of the Invention

The invention relates to a method for controlling an actuator system of a medical device. The invention relates additionally to a signal processing device for carrying out the method, to an operating device for operating a medical device, and to a medical device equipped with such an operating device.

### 2. Discussion of Background Information

A medical device such as, for example, a heat therapy device, for example in the form of an incubator or a heat bed, can be equipped with one or more pedals for controlling an actuator system of the medical device. Such a pedal is generally a movably mounted toggle or pressure switch, which can be correspondingly susceptible to functional impairments as a result of mechanical wear, dirt or other environmental influences.

In view of the foregoing, it would be advantageous to have available an improved method for controlling an actuator system of a medical device. It further would be advantageous to be able to provide a signal processing device for carrying out such a method, a corresponding operating device, and a corresponding medical device.

### SUMMARY OF THE INVENTION

In a first aspect, the invention provides a method for controlling an actuator system of a medical device. The medical device comprises, in addition to the actuator system, an operating device for operating the medical device. The operating device comprises an actuating element, which is actuatable by means of a foot and/or a hand (for example a finger), and a sensor for detecting a deformation of the actuating element. The method comprises: receiving a sensor signal, which indicates a deformation, detected by means of the sensor, of the actuating element on actuation thereof; generating a control signal for controlling the actuator system using the sensor signal.

Such a method allows the actuator to be controlled without movably mounted actuating elements. Mechanical wear is thus reduced. Owing to the lower outlay in terms of construction compared to embodiments with movably mounted actuating elements, the production, maintenance and repair costs can additionally be lowered.

“Deformation” can be understood as meaning in particular (reversible) elastic deformation. The deformation can be determined, for example, on the basis of a (positive or negative) change of at least one of the following electrical quantities, which are detectable by the sensor and can be dependent directly or indirectly on the deformation: a voltage, a current, a resistance. It is possible for the sensor, for example in the form of one or more strain gauges, to be applied with a bias voltage, so that the sensor signal is always positive but either tends toward zero or becomes greater according to the deformation direction.

“Signal”, as in “sensor signal” or “control signal”, can be understood as meaning an analog or digital electrical signal. The control signal can be generated in dependence on, for example, a magnitude, that is to say an intensity, and/or a sign of the sensor signal.

The method can be computer-implemented, for example.

A second aspect, the invention provides a signal processing device. The signal processing device comprises elements which are configured to carry out the method described hereinabove and hereinbelow.

The elements can comprise hardware and/or software modules. In particular, the elements can comprise a processor which is configured to carry out the method (by executing commands of a corresponding computer program). In addition, the elements can comprise a memory and/or a data communication interface for wireless and/or wired data communication with peripheral devices. Alternatively, the signal processing device can be implemented solely as hardware, for example in the form of an ASIC or FPGA module.

For example, the signal processing device can comprise at least one of the following elements: an amplifier for amplifying an analog sensor signal; an analog-digital converter for converting an (optionally amplified) analog sensor signal into a digital sensor signal; a signal analysis unit for analyzing an (optionally amplified) analog sensor signal, for example by filtering and/or Fourier transformation; a processing unit for further processing a digital sensor signal and/or an analog sensor signal (which has optionally been amplified and/or preprocessed by a corresponding signal analysis).

It is possible for at least one of the above-mentioned elements of the signal processing device to be implemented as a hardware and/or software module of the sensor (and vice versa).

It should be noted that features of the method described hereinabove and hereinbelow may also be features of the signal processing device (and vice versa).

In a third aspect, the invention provides an operating device for operating a medical device. The operating device comprises: an actuating element, which is actuatable by means of a foot and/or a hand; a sensor, which is configured to detect a deformation of the actuating element on actuation thereof and to generate a sensor signal, which indicates the detected deformation of the actuating element; a signal processing device as described hereinabove and hereinbelow.

Such an operating device has the advantage that it does not require movably mounted actuating elements. The operating device is therefore correspondingly wear-resistant and robust toward environmental influences such as, for example, mechanical vibrations, dirt, wetness or moisture. In addition, such an operating device requires a lower outlay in terms of construction than embodiments with movably mounted actuating elements. This can facilitate production, maintenance and repair and thus lower the corresponding costs.

The sensor can be, for example, an analog or incremental position sensor (e.g. in the form of a strain gauge), an optical sensor (e.g. in the form of a fiber Bragg grating) or a combination of at least two of these examples.

In a fourth aspect, the invention provides a medical device. The medical device comprises an actuator and an operating device as described hereinabove and hereinbelow.

“Medical device” can be understood as meaning, for example, a heat therapy device for carrying out heat therapy, with a lying surface for a patient. Such a heat therapy device can in particular be in the form of a heat bed, an incubator or a resuscitation unit-for example in each case for a premature or newborn child, an infant or a baby. Alternatively, the medical device can be an operating or work table, a hospital bed, an examination couch, a treatment chair, a diagnostic device (for example a computer or magnetic resonance tomograph) or an X-ray machine.

The actuator system can be formed by one or more actuators, which are controllable by means of the control signal. Such an actuator can be, for example, an electric motor, a solenoid or an electromechanical valve.

Various embodiments of the invention are described in the following. These embodiments are not to be interpreted as limiting the scope of the invention.

According to one embodiment, the operating device can further comprise a feedback device for generating acoustic and/or visual and/or haptic feedback for a (human) user or operator of the medical device.

The feedback device can comprise, for example, at least one of the following components: a light source (e.g. in the form of at least one light-emitting diode, at least one light bulb or at least one, for example elongate, optical fiber) for generating the visual feedback; a display for generating the visual feedback; a loudspeaker for generating the acoustic feedback (e.g. a beep, a sound or a spoken notification); a vibration generator for setting the actuating element in vibration (e.g. in the form of a special electric motor or of a loudspeaker which can be operated with a correspondingly low frequency).

The light source can be configured, for example, to generate the visual feedback by suitably illuminating the medical device itself and/or a floor on which the medical device stands in the operational state, and/or by suitably varying the brightness and/or color of the emitted light.

According to one embodiment, the method can further comprise: generating an additional control signal for controlling the feedback device using the sensor signal and/or the control signal.

It is possible for the feedback device to be activated for a specific period of time in response to the actuation of the actuating element, for example for as long as the sensor signal is being received (with sufficient intensity) and/or the control signal is being generated. “Activation” can be understood as meaning, for example, switching on or alternately switching on and off.

By means of such additional feedback, the ease of use can further be improved.

According to one embodiment, the detected deformation can comprise a detected degree of deformation, wherein the control signal can be generated in dependence on the detected degree of deformation. The detected degree of deformation can be, for example, a magnitude (of an instantaneous amplitude value) of the sensor signal. Alternatively, the detected degree of deformation can be a percentage value, where 0 percent can represent a (for example undeformed) starting state of the actuating element and 100 percent can represent a maximum permissible deformation of the actuating element. This allows the actuator system to be activated in dependence on the actuating force with which the actuating element is currently being actuated (i.e. deformed). To this end, an instantaneous amplitude value can, for example, be assigned to a specific adjustment value from a plurality of possible adjustment values for at least one control parameter for controlling the actuator system—for example an acceleration or speed with which a specific actuator of the actuator system is to be moved—and applied to the control parameter in question.

According to one embodiment, the detected deformation can comprise a detected deformation direction, wherein the control signal can be generated in dependence on the detected deformation direction. The detected deformation direction can be, for example, a sign (of an instantaneous amplitude value) of the sensor signal. This allows the actuator system to be activated in dependence on the actuating direction in which the actuating element is currently being actuated (i.e., deformed). To this end, a sign for the instantaneous adjustment value of the at least one control parameter can be determined, for example, on the basis of the sign of the instantaneous amplitude value and applied to the control parameter in question. In particular, the actuating element can be actuatable in mutually opposite directions.

Alternatively or additionally, the additional control signal (see above) can be generated in dependence on the detected degree of deformation and/or on the detected deformation direction. This allows the feedback to be varied in terms of its type and/or intensity in dependence on the particular actuating force and/or direction, which can have an advantageous impact on the case of use.

According to one embodiment, the sensor can comprise a strain gauge, which is mechanically coupled with at least a portion of the actuating element. “Strain gauge” can be understood generally as meaning a strain sensor for converting a mechanical deformation into an electrical (sensor) signal. The strain gauge can be configured such that an electrical resistance of a material that electrically conductively connects the terminals of the strain gauge to one another changes in dependence on the deformation thereof. Such a strain gauge can be configured, for example, as a planar and/or elongate element (e.g. in the form of a strip or a foil). However, other forms of the strain gauge are also possible (the strain gauge does not necessarily have to be configured as a “strip”). It is expedient for the strain gauge to be applied to a portion of the actuating element that is stretched and/or compressed to a greater extent than the remaining portion of the actuating element on actuation thereof.

According to one embodiment, the sensor signal can indicate as the detected deformation an electric voltage present at the terminals of the strain gauge and/or an electric current flowing between the terminals of the strain gauge. A magnitude of the voltage or current can be dependent on the degree of deformation of the actuating element, and/or a sign of the voltage or current can be dependent on the deformation direction of the actuating element.

According to one embodiment, a deviation of an amplitude of the voltage and/or of the current from a threshold value can be determined, and the control signal can be generated in dependence on the deviation. The deviation can be determined, for example, by subtracting a respective value of the amplitude from the threshold value. “Threshold value” can generally be understood as meaning a (predefined) reference value. The threshold value can be fixed or variable, for example in order to permit fine adjustment of the operating device. The control signal can be generated in particular when the amplitude reaches the threshold value, that is to say the deviation is approaching zero. In this manner, accidental operation can be avoided, for example if the actuating element is inadvertently touched lightly.

According to one embodiment, the sensor can comprise a first sensor element and a second sensor element. The first sensor element can be configured to detect a deformation of a first portion of the actuating element on actuation thereof and to generate a first sensor signal, which indicates the detected deformation of the first portion. The second sensor element can be configured to detect a deformation of a second portion, which is different from the first portion, of the actuating element on actuation thereof and to generate a second sensor signal, which indicates the detected deformation of the second portion. In this case, the signal processing device of the operating device can be configured to generate the control signal using the first sensor signal and/or the second sensor signal. In other words, the control signal can be generated from the first sensor signal, from the second sensor signal or from both sensor signals.

According to one embodiment, receiving the sensor signal can comprise: receiving a first sensor signal, which indicates a deformation, detected by means of the first sensor element, of the first portion of the actuating element on actuation thereof; receiving a second sensor signal, which indicates a deformation, detected by means of the second sensor element, of the second portion of the actuating element on actuation thereof. Accordingly, the control signal can be generated using the first sensor signal and/or the second sensor signal. This allows the deformation to be measured more accurately compared to an embodiment with only one sensor element. A further advantage is that the actuator system can still be controlled even if one of the sensor elements fails.

According to one embodiment, the first sensor element can be a first strain gauge mechanically coupled with the first portion. Accordingly, the first sensor signal can indicate an electric voltage present at the terminals of the first strain gauge and/or an electric current flowing between the terminals of the first strain gauge.

According to one embodiment, the second sensor element can be a second strain gauge mechanically coupled with the second portion. Accordingly, the second sensor signal can indicate an electric voltage present at the terminals of the second strain gauge and/or an electric current flowing between the terminals of the second strain gauge.

The first and second strain gauges can differ from one another in terms of their position and/or orientation relative to the actuating element. The longitudinal axes of the first and second strain gauges can be oriented parallel or obliquely to one another. For example, the longitudinal axes oriented obliquely to one another can enclose an angle of 90 degrees or less, of 60 degrees or less or of 30 degrees or less.

According to one embodiment, the terminals of the first strain gauge can be connected to the terminals of the second strain gauge by way of a bridge circuit, in order to generate a third sensor signal from the first sensor signal and the second sensor signal. In this case, the control signal can be generated using the third sensor signal. This can further improve the reliability and/or accuracy of the method. The third sensor signal can, for example, have a greater amplitude than the first sensor signal and/or the second sensor signal. The bridge circuit can comprise, for example, a full bridge, a half bridge, a quarter bridge or a combination of at least two of these examples.

According to one embodiment, the operating device can further comprise a further actuating element, which is actuatable by means of a foot and/or a hand, and a further sensor for detecting a deformation of the further actuating element. The actuating element and the further actuating element can, for example, be actuatable independently of one another.

According to one embodiment, the method can further comprise: receiving a further sensor signal, which indicates a deformation, detected by means of the further sensor, of the further actuating element on actuation thereof, generating a further control signal for controlling the actuator system using the further sensor signal or using the sensor signal and the further sensor signal. Accidental operation can thus be avoided, for example in that inadvertent simultaneous actuation of the various actuating elements is recognized. For this purpose, a deviation of the sensor signal from the further sensor signal can be determined, for example, wherein the control signal can be generated in dependence on the deviation. It is possible that the same device of the actuator system can be activated with the further control signal as with the control signal. Alternatively, it is possible that a different device of the actuator system can be activated with the further control signal as with the control signal.

According to one embodiment, the actuating element can be plate-like and/or formed of metal. Such an actuating element is particularly robust and/or can be produced particularly easily.

According to one embodiment, the actuating element can be rigidly connectable at its first end to a fastening portion of the medical device and can be actuatable by application of a defined bending force to its second, free end. The actuating element can be rigidly connectable at its first end to the fastening portion by, for example, screwing, welding, soldering, adhesive bonding or a combination of at least two of these connecting methods.

“Fastening portion” can be understood as meaning in particular a portion of a carrying structure of the medical device, for example of a (mobile) frame. Such a fastening portion is generally particularly stiff. This has the effect that, on actuation of the actuating element, it is primarily the fastening element and less so the fastening portion that deforms. On the one hand, this permits particularly precise actuation. On the other hand, greater inaccuracies when detecting the deformation of the actuating element can thus be avoided.

According to one embodiment, at least one (electrical and/or electronic) component of the operating device can be arranged on a printed circuit board, wherein the printed circuit board can be fastened to the actuating element. As a result of being fastened by means of the printed circuit board, the component or components in question can be assembled or disassembled in a single step together with the actuating element, which facilitates assembly or disassembly. In particular, at least one of the following components of the operating device can be arranged on the printed circuit board: the sensor (or at least one component of the sensor), the signal processing device (or at least one component of the signal processing device), the feedback device (or at least one component of the feedback device), a terminal for a power supply.

In addition or alternatively, at least one (electrical and/or electronic) component of the operating device can be arranged on an external printed circuit board, wherein the external printed circuit board can be able to be fastened to a portion of the medical device other than the actuating element, for example to the (mobile) frame thereof.

According to one embodiment, the printed circuit board can be fastened to the actuating element by means of at least one spacer, so that the printed circuit board and the actuating element are separated from one another by a gap. In this case, the at least one component arranged on the printed circuit board can be arranged in the gap. In this manner, the component or components in question can effectively be protected from environmental influences.

According to one embodiment, the gap can additionally be sealed, in order to protect the at least one component arranged in the gap from environmental influences such as, for example, dirt, moisture, wetness, vibrations or electromagnetic radiation. The gap can in particular be sealed in a fluid-tight manner and/or filled at least partly with a suitable sealing material, for example in the form of one or more sealing rings and/or a potting compound (e.g. of synthetic resin and/or silicone).

According to one embodiment, the at least one component arranged on the printed circuit board (and optionally in the gap) can comprise at least one (electrical and/or electronic) component of the feedback device, for example at least one of the following components of the feedback device (see above): the light source, the display, the loudspeaker, the vibration generator. If the light source is arranged in the gap and if the gap is additionally sealed, then it is expedient for the sealing material to be transparent, so that the light emitted by the light source is visible to the user of the medical device from outside.

According to one embodiment, the medical device can be a heat therapy device. The heat therapy device can be in the form of, for example, a heat bed, an incubator, a resuscitation unit or a combination of at least two of these examples (e.g. in each case for a premature or newborn child, an infant or a baby).

According to one embodiment, the medical device can further comprise a lying surface for a patient. In this case, the actuator system can comprise a lying surface adjustment device, which is controllable by the signal processing device of the operating device, for moving the lying surface, for example in order to adjust its position and/or orientation relative to the floor on which the medical device is standing. “Lying surface” can be understood as meaning, for example, a (heatable) lying deck. The lying surface adjustment device can comprise, for example, one or more lifting columns and/or a rocker-like mounting of the lying surface.

According to one embodiment, the medical device can further comprise an incubator chamber for accommodating a premature or newborn child. In this case, the actuator system can comprise at least one of the following devices, which can be controllable by the signal processing device of the operating device: a chamber adjustment device for moving the incubator chamber, for example in order to adjust its position and/or orientation relative to the floor on which the medical device is standing; a hood adjustment device for moving a hood of the incubator chamber, for example in order to open and/or close the incubator chamber by means of the hood. The chamber adjustment device can comprise, for example, one or more lifting columns for adjusting the incubator chamber. The incubator chamber can be heatable. In addition or alternatively, the humidity inside the incubator chamber can be controllable.

According to one embodiment, the medical device can further comprise a mobile frame having a plurality of castors for moving the medical device. In this case, the actuator system can comprise at least one of the following devices, which can be controllable by the signal processing device of the operating device: a drive device for driving at least one of the castors; a braking device for braking at least one of the castors (for example in the form of an electric parking brake); a steering device for steering at least one of the castors.

“Braking” and/or “steering” can also be understood as meaning locking in one or more directions.

### BRIEF DESCRIPTION OF THE DRAWINGS

Embodiments of the invention will be described in the following with reference to the accompanying drawings. Neither the description nor the drawings are to be interpreted as limiting the scope of the invention.

FIG. 1 shows an operating device according to an embodiment of the invention.

FIG. 2 shows an actuating element from FIG. 1 from beneath.

FIG. 3 shows a medical device according to an embodiment of the invention.

FIG. 4 shows a portion of a mobile frame of a medical device according to an embodiment of the invention.

FIG. 5 shows a portion of the mobile frame from FIG. 4 from beneath.

The drawings are purely schematic and are not true to scale. If the same reference signs are used in different drawings, then these reference signs denote the same features or features having the same effect.

### DETAILED DESCRIPTION OF EXEMPLARY EMBODIMENTS

The particulars shown herein are by way of example and for purposes of illustrative discussion of the embodiments of the present invention only and are presented in the cause of providing what is believed to be the most useful and readily understood description of the principles and conceptual aspects of the present invention. In this regard, no attempt is made to show details of the present invention in more detail than is necessary for the fundamental understanding of the present invention, the description in combination with the drawings making apparent to those of skill in the art how the several forms of the present invention may be embodied in practice.

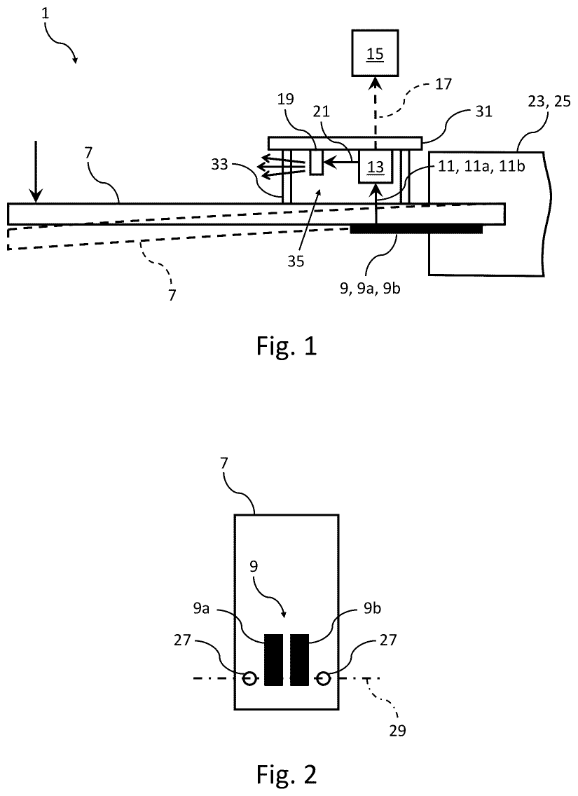

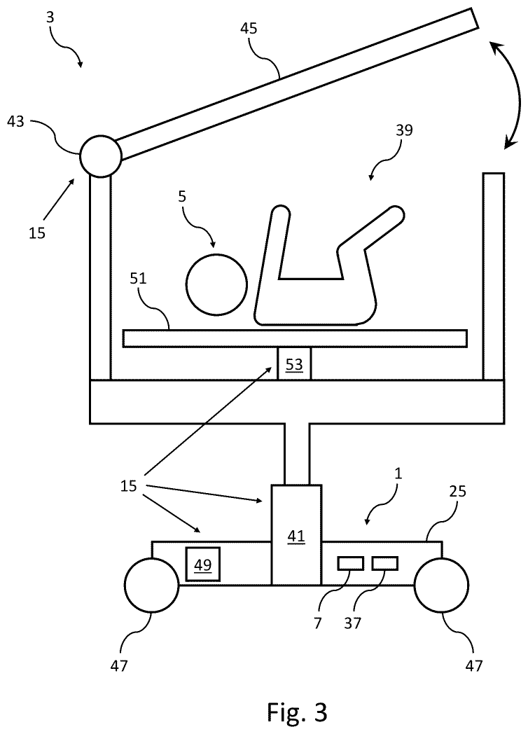

FIG. 1 shows an operating device 1 for operating a medical device 3, here an incubator 3 for a premature or newborn child 5 (see FIG. 3). The operating device 1 comprises an actuating element 7, which is actuatable by means of a foot and/or a hand (for example a finger), a sensor 9, which is configured to detect a (predominantly elastic) deformation of the actuating element 7 on actuation thereof and to generate an analog or digital electrical sensor signal 11, which indicates the detected deformation of the actuating element 7, and a corresponding signal processing device 13.

The signal processing device 13 comprises elements which are configured to carry out the following method for controlling an actuator system 15 of the incubator 3.

In a first step of the method, the sensor signal 11 is received in the signal processing device 13. Then, in a second step of the method, a control signal 17 for controlling the actuator system 15 is generated.

The actuator system 15 can comprise, for example, at least one of the following actuators, which are controllable by the signal processing device 13: an electric motor, a solenoid, an electromechanical valve.

The elements of the signal processing device 13 can comprise, for example, a processor and a memory. In this case, the processor can be configured to carry out the method by executing a computer program stored in the memory.

The operating device 1 can additionally comprise a feedback device 19 for generating acoustic and/or visual and/or haptic feedback for a user of the incubator 3. In this case, an additional control signal 21 for controlling the feedback device 19 can be generated in an additional step of the method using the sensor signal 11 and/or the control signal 17.

The feedback device 19 can comprise, for example, at least one of the following components: a light source (e.g. in the form of at least one light-emitting diode, at least one light bulb or at least one, for example elongate, optical fiber) for generating the visual feedback; a display for generating the visual feedback; a loudspeaker for generating the acoustic feedback (e.g. a beep, a sound or a spoken notification); a vibration generator for setting the actuating element 7 in vibration (e.g. in the form of a special electric motor or of a loudspeaker which can be operated with a correspondingly low frequency). The light source can be configured, for example, to generate the visual feedback by illuminating a floor on which the incubator 3 stands in the operational state with a suitable pattern, and/or by suitably varying the brightness and/or color of the emitted light.

The additional control signal 21 can be generated, for example, as a (direct) response to the receiving of the sensor signal 11 and/or to the generation of the control signal 17. It is possible for the additional control signal 21 to be generated for as long as the sensor signal 11 is being received (with sufficient intensity) and/or the control signal 17 is being generated.

The sensor signal 11 can indicate a detected degree of deformation and/or a detected deformation direction (indicated in FIG. 1 by a downwardly directed vertical arrow). Accordingly, the control signal 17 can be generated in dependence on the detected degree of deformation and/or on the detected deformation direction. This allows the actuator system 15 to be controlled in dependence on the particular actuating force and/or direction.

The detected degree of deformation can correspond, for example, to an instantaneous magnitude of the sensor signal 11, that is to say its instantaneous intensity. Alternatively, the detected degree of deformation can be a percentage value between 0 (for a, for example undeformed, starting state of the actuating element 7) and 100 (for maximum permissible deformation of the actuating element 7). The detected deformation direction can correspond, for example, to an instantaneous positive or negative sign of the sensor signal 11, wherein each of the signs can indicate one of two mutually opposite deformation directions.

The control signal 17 can be generated, for example, in dependence on a deviation of an amplitude of the sensor signal 11 from a given fixed or variable threshold value, in particular only when the amplitude reaches or exceeds the threshold value. In this manner, accidental operation can be avoided, for example if the actuating element 7 is inadvertently pressed lightly.

As can be seen in FIG. 2, the sensor 9 can comprise a first sensor element 9a for detecting a deformation of a first portion of the actuating element 7 and a second sensor element 9b for detecting a deformation of a second portion, which is different from the first portion, of the actuating element 7. The sensor 9 can also comprise more than two such sensor elements.

Accordingly, there can be received in the signal processing device 13 a first sensor signal 11a, which indicates a deformation, detected by means of the first sensor element 9a, of the first portion of the actuating element 7 on actuation thereof, and a second sensor signal 11b, which indicates a deformation, detected by means of the second sensor element 9b, of the second portion of the actuating element 7 on actuation thereof. The control signal 17 can then be generated using the first sensor signal 11a and/or the second sensor signal 11b. This allows more accurate measurement of the deformation compared to an embodiment with only one sensor element. A further advantage is that the actuator system 15 can still be controlled even if one of the sensor elements 9a, 9b fails.

The sensor elements 9a, 9b can each be configured as a strain gauge, which is mechanically coupled with (for example adhesively bonded to) the respective portion of the actuating element 7.

Accordingly, the sensor signals 11a, 11b can each indicate an electric voltage present at the terminals of the respective strain gauge and/or an electric current flowing between the terminals of the respective strain gauge.

The different strain gauges can differ from one another in terms of their position and/or orientation relative to the actuating element 7. The respective longitudinal axes of the strain gauges can be oriented parallel—as shown by way of example in FIG. 2—or obliquely to one another. For example, the longitudinal axes oriented obliquely to one another can enclose an angle of 90 degrees or less, of 60 degrees or less or of 30 degrees or less.

The terminals of the different strain gauges can optionally be connected to one another by way of a bridge circuit. The bridge circuit can be configured to generate a third sensor signal from the first sensor signal 11a and the second sensor signal 11b. The third sensor signal can, for example, have a greater amplitude than each of the two other signals 11a, 11b. The bridge circuit can comprise, for example, a full bridge, a half bridge, a quarter bridge or a combination of at least two of these examples. The control signal 17 can then be generated using the third sensor signal. This can further improve the reliability and/or accuracy of the method.

In the example shown in FIG. 1 and FIG. 2, the sensor elements 9a, 9b have each been applied to an underside of the plate-like actuating element 7. The actuating element 7 is in this case compressed, that is to say subjected to pressure, to a particularly great extent when it is actuated. Other locations are also possible, for example a location on an upper side of the actuating element 7 opposite the underside. The actuating element 7 is there stretched, that is to say subjected to tension, to a particularly great extent when it is actuated.

As is shown in FIG. 1, the actuating element 7 can be rigidly connected at its first end to a fastening portion 23 of the incubator 3. The actuating element 7 can then be actuated, that is to say bent (the bent state is indicated by broken lines), by application of a vertical force to its free, second end.

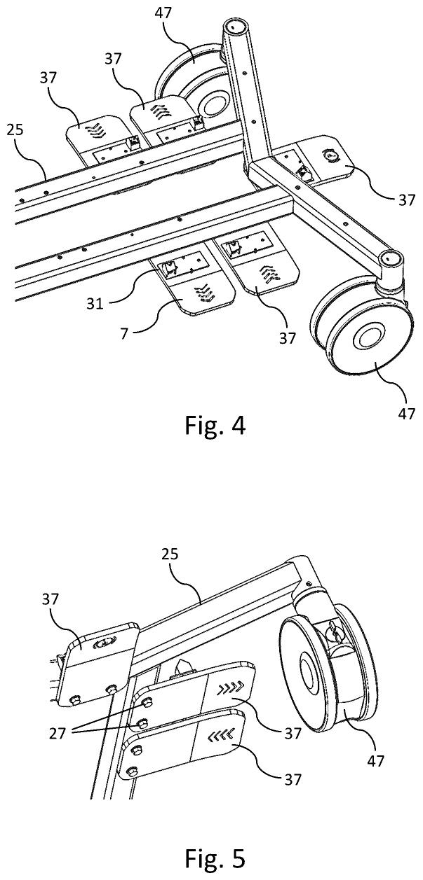

In this example, the actuating element 7 is screwed by way of two screws 27 to a portion of a mobile frame 25 (see also FIG. 3 to FIG. 5) as the fastening portion 23 and is deformable by being pressed down by means of a foot. Alternatively or in addition to the screws 27, the actuating element 7 can be fastened to the fastening portion 23 by means of, for example, welding, soldering and/or adhesive bonding.

As can be seen in FIG. 2, the two sensor elements 9a, 9b can be arranged such that they overlap a notional straight connecting line 29 between the two screws 27. The actuating element 7 is generally deformed to the greatest extent in this region when it is actuated.

In order to facilitate assembly and disassembly, the signal processing device 13 and the feedback device 19 are in this example arranged on a common printed circuit board 31, which is fastened by way of a plurality of spacers 33 to the upper side of the actuating element 7.

The signal processing device 13 and the feedback device 19 are here located in a gap 35 between the printed circuit board 31 and the actuating element 7 and are thus well protected from environmental influences. In addition, the gap 35 can be closed in a dust-and/or water-tight manner.

As is indicated in FIG. 1, the feedback device 19 can, for example, be configured to illuminate the gap 35, wherein part of the emitted light is able to pass through to the outside, so that the user can be given visual feedback on actuation of the actuating element 7.

As is shown in FIG. 3 to FIG. 5, the operating device 1 can comprise one or more further actuating elements 37 each having a further sensor for detecting a deformation of the respective further actuating element. The further actuating elements 37 and the further sensors can be configured analogously to the actuating element 7 described above and the sensor 9 thereof.

In this case, by using a further sensor signal which indicates a deformation, detected by means of one of the further sensors, of one of the further actuating elements on actuation thereof, a further control signal for controlling the actuator system 15 can be generated, for example by a corresponding further signal processing device analogous to the signal processing device 13 described above.

The further control signal can be generated, for example, with the additional use of the sensor signal 11 or of at least one of the sensor signals 11a, 11b. Conversely, it is possible for the control signal 17 to be generated with the additional use of the further sensor signal or of the further sensor signals. This allows inadvertent simultaneous actuation of the different actuating elements 7, 37 to be recognized.

As is shown in FIG. 3, the incubator 3 can comprise an incubator chamber 39 for accommodating the premature or newborn child 5. In this case, the actuator system 15 can comprise, for example, an electrically controllable chamber adjustment device 41 for moving the incubator chamber 39 (as a whole) and/or an electrically controllable hood adjustment device 43 for opening and/or closing a displaceably and/or pivotably mounted hood 45 of the incubator chamber 39.

The mobile frame 25 can comprise a plurality of castors 47 for moving the incubator 3 on a floor. In this case, the actuator system 15 can comprise, for example, an electrically controllable drive and/or braking and/or steering device 49 for driving and/or braking and/or steering at least one of the castors 47.

In addition or alternatively, the incubator 3 can comprise an adjustable lying surface 51 for the premature or newborn child 5. In this case, the actuator system 15 can comprise an electrically controllable lying surface adjustment device 53 for adjusting the lying surface 51, for example the inclination and/or height thereof. The lying surface 51 and the incubator chamber 39 can, for example, be adjustable independently of one another.

Finally, it should be noted that terms such as “have”, “comprise”, “include”, “with”, etc. do not exclude other elements or steps, and indefinite articles such as “a” and “an” do not exclude a plurality.

It should further be noted that features or steps that are described with reference to one of the above embodiments may also be used in combination with features or steps that are described with reference to other of the above embodiments.

### LIST OF REFERENCE NUMERALS

1 operating device
3 medical device, incubator
5 premature or newborn child
7 actuating element
9 sensor
9a first sensor element
9b second sensor element
11 sensor signal
11a first sensor signal
11b second sensor signal
13 signal processing device
15 actuator system
17 control signal
19 feedback device
21 additional control signal
23 fastening portion
25 mobile frame
27 screw
29 connecting line
31 printed circuit board
33 spacer
35 gap
37 further actuating element
39 incubator chamber
41 chamber adjustment device
43 hood adjustment device
45 hood
47 castor
49 drive and/or braking and/or steering device
51 lying surface
53 lying surface adjustment device

What is claimed is:

1. A method for controlling an actuator system of a medical device, the medical device comprising, in addition to the actuator system, an operating device for operating the medical device, the operating device comprising an actuating element, which is actuatable by means of a foot and/or a hand, and a sensor for detecting a deformation of the actuating element, wherein the method comprises:
receiving a sensor signal, which indicates a deformation, detected by the sensor, of the actuating element on actuation thereof;
generating a control signal for controlling the actuator system using the sensor signal.

2. The method of claim 1, wherein the operating device further comprises a feedback device for generating acoustic and/or visual and/or haptic feedback for a user of the medical device;
wherein the method further comprises:
generating an additional control signal for controlling the feedback device using the sensor signal and/or the control signal.

3. The method of claim 2, wherein the detected deformation comprises a detected degree of deformation, and the control signal is generated in dependence on the detected degree of deformation; and/or
wherein the detected deformation comprises a detected deformation direction, and the control signal is generated in dependence on the detected deformation direction; and/or
wherein the sensor comprises a strain gauge, which is mechanically coupled with at least a portion of the actuating element, and the sensor signal indicates as the detected deformation an electric voltage present at terminals of the strain gauge and/or an electric current flowing between terminals of the strain gauge.

4. The method of claim 3, wherein a deviation of an amplitude of the voltage and/or of the current from a threshold value is determined, and the control signal is generated in dependence on the deviation.

5. The method of claim 1, wherein receiving the sensor signal comprises:
receiving a first sensor signal, which indicates a deformation, detected by a first sensor element of the sensor, of a first portion of the actuating element on actuation thereof;
receiving a second sensor signal, which indicates a deformation, detected by a second sensor element of the sensor, of a second portion, which is different from the first portion, of the actuating element on actuation thereof;
the control signal being generated using the first sensor signal and/or the second sensor signal.

6. The method of claim 1, wherein the operating device further comprises a further actuating element, which is actuatable by a foot and/or a hand, and a further sensor for detecting a deformation of the further actuating element);
wherein the method further comprises:
receiving a further sensor signal, which indicates a deformation, detected by the further sensor, of the further actuating element on actuation thereof;
generating a further control signal for controlling the actuator system using the further sensor signal or using the sensor signal and the further sensor signal.

7. A signal processing device, wherein the device comprises elements which are configured for carrying out the method of claim 1.

8. An operating device for operating a medical device, wherein the operating device comprises:
an actuating element, which is actuatable by a foot and/or a hand;
a sensor, which is configured to detect a deformation of the actuating element on actuation thereof and to generate a sensor signal, which indicates the detected deformation of the actuating element;
the signal processing device of claim 7.

9. The operating device of claim 8, wherein the actuating element is plate-like and/or formed of metal; and/or wherein the actuating element is rigidly connectable at its first end to a fastening portion of the medical device and is actuatable by application of a defined bending force to its second, free end.

10. The operating device of claim 8, wherein at least one component of the operating device is arranged on a printed circuit board, wherein the printed circuit board is fastened to the actuating element.

11. The operating device of claim 10, wherein the printed circuit board is fastened to the actuating element by at least one spacer, so that the printed circuit board and the actuating element are separated from one another by a gap, the at least one component arranged on the printed circuit board being arranged in the gap.

12. The operating device of claim 11, wherein the gap is additionally sealed, in order to protect the at least one component arranged in the gap from environmental influences.

13. The operating device of claim 10, wherein the device further comprises:
a feedback device for generating acoustic and/or visual and/or haptic feedback for a user of the medical device;
the elements of the signal processing device being configured for carrying out a method for controlling an actuator system of a medical device, the medical device comprising, in addition to the actuator system, an operating device for operating the medical device, the operating device comprising an actuating element, which is actuatable by means of a foot and/or a hand, and a sensor for detecting a deformation of the actuating element, the method comprising:
receiving a sensor signal, which indicates a deformation, detected by the sensor, of the actuating element on actuation thereof;
generating a control signal for controlling the actuator system using the sensor signal,
and the operating device further comprising a feedback device for generating acoustic and/or visual and/or haptic feedback for a user of the medical device and the method further comprising generating an additional control signal for controlling the feedback device using the sensor signal and/or the control signal;
and wherein the at least one component arranged on the printed circuit board comprises at least one component of the feedback device.

14. A medical device, wherein the device comprises:
an actuator system; and
the operating device of claim 8.

15. The medical device of claim 14, wherein the medical device is a heat therapy device.

16. The medical device of claim 14, wherein the medical device further comprises a lying surface for a patient, the actuator system comprising a lying surface adjustment device, which is controllable by the signal processing device of the operating device, for moving the lying surface.

17. The medical device of claim 14, wherein the medical device further comprises an incubator chamber for accommodating a premature or newborn child, the actuator system comprising at least one of the following devices, which are controllable by the signal processing device of the operating device: a chamber adjustment device for moving the incubator chamber; a hood adjustment device for moving a hood of the incubator chamber.

18. The medical device of claim 14, wherein the medical device further comprises a mobile frame having a plurality of castors for moving the medical device, the actuator system comprising at least one of the following devices, which are controllable by the signal processing device of the operating device: a drive device for driving at least one of the castors; a braking device for braking at least one of the castors; a steering device for steering at least one of the castors.
